(19) United States
(12) Patent Application Publication
(10) Pub. No.: US 2006/0058206 A1
(43) Pub. Date: Mar. 16, 2006

(54) Aqueous activated components conveyed in a non-aqueous carrier system

(75) Inventors: John E. Walls, FORT COLLINS, CO (US); Jeffrey W. Putt, Fort Collins, CO (US); Kenneth E. Deline, Fort Collins, CO (US)

Correspondence Address: CR Miles, 1 Old Town Square, Suite 200 B, Fort Collins, CO, 80524, US

(73) Assignee: VENTURE MANAGEMENT ALLIANCE, GREELEY, CO (US)

(21) Appl. No.: 10/533,262

(22) PCT Filed: Nov. 3, 2003
(86) PCT No.: PCT/US03/35057

§ 371 Date: Apr. 27, 2005

Related U.S. Application Data
(60) Provisional application No. 60/423,231, filed on Nov. 2, 2002.

(51) Int. Cl. A61K 8/00

(52) U.S. Cl. 510/130

(57) ABSTRACT

Generally, compositions that provide, either individually or in combination, aqueous activated components conveyed in a non-aqueous carrier system, perceivable sensorial indicia of discrete event occurrence, or perceivable sensorial reinforcers to provide sensorial incentives or sensorial disincentives, to encourage or discourage, attainment of discrete event occurrence.

This International Patent Cooperation Treaty Patent Application claims the benefit of U.S. Provisional Patent Application No. 60/423,231, filed Nov. 2, 2002, hereby incorporated by reference.

### I. TECHNICAL FIELD

Generally, aqueous activated components conveyed in a non-aqueous carrier system that can further provide perceivable sensorial indicia of discrete event occurrence, or perceivable sensorial reinforcer(s) as sensorial incentives or sensorial disincentives, to encourage or to discourage, attainment of a discrete event occurrence.

Specifically, a hand washing system that provides a non-aqueous carrier system to convey aqueous activated components to the surface of hands and can further provide one or more perceived sensorial indicia to provide notice to the hand washer that a desired duration or level of hand washing has been achieved, or perceivable sensorial reinforcer(s) as sensorial incentives to encourage attainment of the desired duration or level of hand washing.

### II. BACKGROUND

Conventional cosmetics, including hand-washing agents, can comprise a carrier which conveys one or more compositions that provide conventional perceivable sensorial attributes. A common sensorial attribute can be a fragrance. Pleasing odor can induce persons to purchase and re-purchase the cosmetic. The manner in which the fragrance is released can also be an important factor influencing a person's physiology and psychology as described by U.S. Pat. No. 5,238,915.

Another common sensorial attribute can be color. Colorants can be added to cosmetic products to render them consistent in color or can be added to cosmetic products in a manner to create decorative patterns or to differentiate portions of the cosmetic product as described in U.S. Pat. No. 6,147,040, or International Patent Application No. WO 99/40172. Certain cosmetic products are provided with an initial colorant or a secondary colorant within capsules which generate color or color changes of the initial colorant upon rupture of the capsules as described by U.S. Pat. Nos. 4,756,906 and 5,320,835.

Sometimes the sensorial attribute is that of temperature. Coolness can be imparted to cosmetic products such as toothpastes or aftershave lotions through the presence of xylitol, camphor, menthol or menthol derivatives such as menthol lactate as described by International Patent Application WO 01/12148 or U.S. Pat. No. 5,861,440.

Other conventional cosmetic products provide exothermic temperature increase by inclusion of capsaicin or aluminosilicate interacting with water, each providing a brief exothermic reaction as a source of heat as described by U.S. Pat. Nos. 4,379,143; 4,626,550; 4,362,715; or 3,250,680. Certain cosmetic products provide a two-stage temperature rise, such as, the two-stage chemically heated soap composition described by U.S. Pat. No. 4,839,081. The temperature rise may be accompanied by exothermic effervescence which in itself may be appealing, or may assist to disperse other perceivable sensorial attributes, such as fragrance, as described by U.S. Pat. No. 5,993,854.

Feel of a product can be highly important. Conventional creams, lotions, gels or pastes can be adjusted to provide the desired tactility. A graded series of tactility from silky to tacky can be provided as disclosed by International Patent Application WO 01/12148.

While each of these conventional perceived sensorial attributes may be added to conventional cosmetic compositions to address the concerns of personal taste, perception of these sensorial attributes may not be coupled to coincide with occurrence of one or more discrete events to provide notice to the user such discrete event has occurred, such as therapeutic efficacy of the product, or attainment of an established guideline for product use. Nor do conventional perceived sensorial attributes provide perceivable sensorial reinforcers as incentives to encourage or discourage product use to achieve or avoid coincidence with the occurrence of discrete event(s), such as attainment of a duration of product use or avoidance of excess product use.

As but one non-limiting example, while conventional hand washing agents or compositions can include one, or more than one, conventional perceived sensorial attributes such as color, fragrance, or tactility, none of these perceived sensorial attributes provide indicia as to when efficacy of the particular hand washing event has been achieved, or when compliance with hand washing procedure(s) or guideline(s) has been met. Additionally, none of these conventional perceived sensorial attributes are utilized to increase the probability that the user will be compliant with such hand washing procedure(s) or guideline(s).

As such, while conventional hand washing procedures utilizing soap and water may the single most effective practice that prevents the spread of germs, and experts unequivocally agree that conventional hand washing with soap and water can reduce the incidence of nosocomial infections, compliance with hand washing guidelines is embarrassingly and unacceptably low. In fact, studies have consistently demonstrated that rates of hand washing compliance is less than 50% as described by “A Survey On Handwashing Practices And Opinions Of Healthcare Workers”, Journal of Hospital Infection, 45(4): 318-321 (2000). This failure to comply with hand washing guidelines may contribute to nosocomial infections which have been shown to complicate between six and fourteen percent of admissions in pediatric institutions. Posfay-Barbe, Kiara and Pittet, Didier, “New Concepts In Hand Hygiene”, W.B. Saunders Company, (2001). These hospital infections are the fourth leading cause of death. Many of the deaths were caused by unsanitary facilities, germ laden instruments, and unwashed hands. “Hospital Infections Fourth Leading Cause of Death”, The Associated Press, The Coloradoan, p. A3 (Jul. 21, 2002).

Similarly, many millions of non-health care workers get sick with colds, flu or stomach viruses that could be prevented by proper hand washing. The Center for Disease Control (CDC) estimates that nearly 22 million school days are lost each year due to colds alone, and that 75% of those colds result in visits to the doctor's office. In addition, the CDC reports that, out of the 95 million cases of influenza reported annually, 70-million work-loss days result. The CDC states that hand washing is “the single most effective practice that prevents the spread of germs”.

Because of the prevalence of the problem and the size of the commercial market in which hand washing products can be sold, a variety of conventional technologies have been proposed to improve hand washing compliance. These include devices used when hands are washed, such as, automated sinks with water flow and soap dispensing controlled by electronic sensors. Larson, E., McGeer A., Quarisi, A., et al., “Effect of an automated sink on handwashing practices and attitudes in high-risk units”, Infec. Control Hosp. Epdemiol. 12:442-448 (1991). This conventional technology can further include hand washing machines. Decker, L., Gross, A., Miller, Read, J., Cutright, D. and Devine, J., “A Rapid Method For The Presurgical Cleansing Of Hands”, Obstet. Gynecol. 51:115-117 (1978). Certain conventional technologies also include soap dispensers with timed signaling activated at the time soap is dispensed which signals the user after a predetermined interval indicating that the washing time is over as disclosed by U.S. Pat. No. 5,771,925.

Even though numerous conventional technologies have been developed to promote the proper application and use of cosmetic agents in general, and specifically to meet procedural guidelines or regulations with respect to hand washing, a number of problems remain unresolved with respect to conventional cosmetic technologies in general, and with respect to hand washing agents specifically.

A significant problem with conventional cosmetic technologies can be that while the cosmetic user understands the importance of proper application and use of a cosmetic agent, they tend to overestimate their own compliance with procedures or guidelines. With respect to hand washing, as an example, healthcare workers understand the importance of hand washing, but they tend to overestimate compliance with hand washing guidelines. “Effectiveness Of A Hospital-Wide Program To Improve Compliance With Hand Hygiene”, Lancet, 356 (9238): 1307-1312 (2000). Therefore, even when various conventional technologies are made available they may not be used because users may incorrectly believe they are all ready using the cosmetic agent properly, or in compliance with guidelines, such as hand washing guidelines.

Another significant problem with conventional cosmetic technologies can be cosmetic user distaste for external intervention. For example, healthcare workers are not in favor of interventions involving rewards and punishments to achieve hand washing compliance. Pittet, D., “Effectiveness Of A Hospital-Wide Program To Improve Compliance With Hand Hygiene”, Lancet, 356 (9238): 1307-1312 (2000). As such, technologies which provide portable, individualized, hand washing agent dispensers which track and store into memory the site where workers dispense hand wash agents as described by U.S. Pat. No. 6,392,546; or the use of modules at wash sinks into which employees input identification codes to track compliance, may actually reduce compliance or may be disfavored by employees.

Another significant problem with conventional cosmetic technology may be that ineffective or less effective conventional cosmetic technologies are selected when they are easier to use. Healthcare workers are attracted to interventions that make hand washing easier. Pittet, D., “Effectiveness Of A Hospital-Wide Program To Improve Compliance With Hand Hygiene”, Lancet, 356 (9238): 1307-1312 (2000). As such, heathcare workers may become reliance on pre-moistened towelettes, waterless hand cleaners, or wipes. United States Department of Health and Human Services, Center For Disease Control, The ABC's of Safe and Healthy Child Care (2002). However, pre-moistened towelettes do not effectively clean and do not take the place of hand washing. Waterless hand cleaners or pre-moistend towelettes should not be used as a substitute for washing hands with soap and water. Towelettes should only be used to remove residue, such as food off a baby's face or feces from a baby's bottom during diaper changing.

Another significant problem with conventional cosmetic technology may be that alternative cosmetics used for the same or similar procedure may have different performance characteristics. For example, various types of antiseptic ingredients included in cosmetic compositions, may have different performance characteristics. A first characteristic of antiseptic agents which may differ can be duration of time to achieve maximum reduction in bacteria counts. Alcohol based preparations, for example, require less time to effect maximum reduction in bacterial counts than did, for example, a product containing chlorhexidine gluconate (CHG). Indeed, a one-minute immersion or scrub with alcohol is as effective as a four to seven minute skin preparation with other antiseptics. Hexachlorophene on the other hand may not be particularly fast acting and its rate of killing is slow to intermediate. Larson, Elaine, “APIC Guideline For Hand Washing And Hand Antisepsis in Health-Care Settings” (1995). A second characteristic of antiseptic agents which may differ can be their ability to bind to the stratum corneum, resulting in a persistent activity on the skin. For example, CHG may be effected by individual differences in skin pH, secretions, or moisture level. A third characteristic of antiseptic agents which may differ can be concentration to achieve microbiocidal activity. For example, free iodine is the major chemical and microbiocidal factor in the activity of iodophors and changes with the degree of dilution.

Perhaps common to each of these prominent problems with conventional cosmetic technology may be the lack of any perceivable sensorial indicia generated by the cosmetic agent to inform the user of occurrence of discrete events, such as achievement of a standard for compliance with procedures that utilize a particular cosmetic, or attainment of therapeutic efficacy with the cosmetic. As such, provision of feed back to the user of conventional cosmetics involves additional mechanical timing or signaling devices, activity recording or retrieval devices, or the like, some of which are described above in the context of hand washing compliance.

Additionally, conventional cosmetic technology may not provide perceivable sensorial reinforcement to increase the probability, or decrease the probability, that cosmetic product use will be coincident with occurrence of a discrete event. For example, health care workers can be induced to perform hand washing from the use of strategically placed reminders, or asking patients to remind staff of the need to conduct hand washing, each having a positive effect on compliance. Naikoba, S. and Hayward, A., “The Effectiveness Of Interventions Aimed At Increasing Handwashing In Healthcare Workers—A Systematic Review”, The Hospital Infection Society 0195-6701/01/030173+08. However, these types of reinforcement require the intervention of other devices or persons which may not always be available, reliable or consistent.

A prominent problem within the broad context of conventional cosmetic compositions, and specifically with regard to providing perceivable sensorial indicia or providing perceivable sensorial reinforcers as a solution to problems of compliance with hand washing procedures, may be that cosmetic carriers cannot contain or act as the conveyance for components, elements, or compositions activated by water. One aspect of this problem can be that the carrier itself may contain sufficient water to activate such aqueous activated components. As such, aqueous activated components cannot reside in or be conveyed by the carrier for the desired length of time without being activated by the carrier itself. A second aspect of this problem may be that while the carrier does not activate aqueous activated components or elements, it is not water miscible. This may preclude the carrier from being used in applications in which the carrier and water must readily form a single phase. A third aspect of this problem may be that the carrier may interfere with the activation, dissemination, attributes, or perceivable indicia of the aqueous activated components it conveys. A fourth aspect of this problem may be that non-aqueous carriers are not suitable for application to the skin.

The present invention provides a cosmetic system in which aqueous activated components can be conveyed in a non-aqueous carrier and methods of use which provide a solution to each of the above-mentioned problems.

### III. DISCLOSURE OF INVENTION

Accordingly, a broad object of the invention can be to provide a cosmetic carrier(s) for a desired application(s) which further acts as suitable conveyance for components that remain dormant until the product is utilized, or a particular step commences, or a specific time duration elapses. One aspect of this object of the invention can be to provide a non-aqueous carrier that holds aqueous activated components dormant, or provides conveyance of dormant aqueous activated components to the site or surface where combination with aqueous activating agents occurs. A specific aspect of this objective can be to provide a non-aqueous but water miscible carrier for the conveyance of aqueous activated components to the surface of the human skin, hair, or nails.

Another broad object of the invention can be to provide cosmetics that contain perceivable sensorial indicia (the term “perceivable sensorial indicia” is intended to encompass any manner of indicator perceivable by the human senses generated by the invention coincident with occurrence of a discrete event, not the generation of the indicator itself). As non-limiting examples, certain hand washing agents have a concentration of iodophore at the beginning of handwashing procedure which may not have, or have less than, the desired efficacy. By providing perceivable sensorial indicia of efficacious iodophore concentration, the hand washer can understand when the level of iodophore present during a hand washing procedure is sufficient to reduce bacterial populations. Also, cosmetics that provide perceivable sensorial indicia provide the user notice of when to stop product use. This can be particularly useful when the cosmetic has a utility without an otherwise sensorial perceivable end point, such a therapeutic efficacy or elapse of a duration of time; or when the cosmetic application requires performance of a plurality of different steps, or when the cosmetic requires serial timed application; or when one or more cosmetic agents need to be mixed to achieve a desired result.

Another broad object of the invention can be to provide a perceivable sensorial reinforcer (the term “perceivable sensorial reinforcer” is intended to encompass one or more attributes of the cosmetic perceivable by the human senses which can increase or decrease the probability of coincidence between cosmetic use and occurrence of a discrete event. The various approaches to achieving this object of the invention can be divided among the traditional reinforcement schedules or operant conditioning theories, if desired, to increase the probability of attaining a discrete event or to decrease the probability of a attaining a discrete event. As such, embodiments of sensorial reinforcer can include both, individually or in combination, the manner of releasing the perceivable sensorial indicia, or provision of a discrete perceivable sensorial reinforcer as incentive to achieve generation of the perceivable sensorial indicia, or provision of a discrete perceivable sensorial reinforcer released as a reward for achieving generation of perceivable sensorial indicia.

As a non-limiting example, an embodiment of the invention can provide a hair conditioner that requires an amount of mechanical manipulation to achieve a desired degree of efficacy can include a perceivable sensorial indicia, such as color change, to indicate that the desired amount of mechanical manipulation has occurred. A further discrete perceivable sensorial reinforcement can provide reward for achieving the generation of color by release of fragrance. Alternately, for example, the manner of releasing the perceivable sensorial indicia can be adjusted so that color intensity increases until the discrete event has occurred.

Another broad object of the invention can be to provide hand washing agents having a non-aqueous carrier that contain aqueous activated perceived sensorial indicia, or provide aqueous activated components that develop or release perceivable sensorial indicia after hand washing has begun. The perceivable sensorial indicia can be selected to generate indicators such as color not the color of the hand washing agent prior to hand washing; a first color and a second color neither of which are the color of the hand washing agent prior to hand washing; a first color, a second color, and a third color none of which are the color of the hand washing agent prior to hand washing; an audible sound having a decibel level greater than typically generated by hand washing with a hand washing agent; a first fragrance not the fragrance of the hand washing agent prior to hand washing; a first and a second fragrance neither of which are the fragrance of the hand washing agent prior to hand washing; or a tactility perceivable only after hand washing has begun, or the like.

Another broad object of the invention can be to provide hand washing agents that develop or release perceivable sensorial indicia upon completion of a desired hand washing duration, or attainment of a level of hand washing having a desired degree of therapeutically efficacy. As to these embodiments of the invention, the perceivable sensorial indicia can be selected to generate indicators such as at least one color not perceivable until the discrete event occurs; color intensity not perceivable until the discrete event occurs; a first color and a second color neither of which are perceivable until the discrete event occurs; a first color, a second color, and a third color, none of which are the perceivable until the discrete event occurs; a first color and a second color, the first color perceivable after hand washing beings and the second color not perceivable until the discrete event occurs; a first color, a second color, and a third color, the first and second color perceivable serially after hand washing begins, the third not perceivable until the discrete event occurs; an audible sound having a decibel level greater than typically generated by use of hand washing agents not perceivable until the discrete event occurs; a first audible sound, and a second audible sound having perceivably greater decibel value than the first audible sound, the first audible sound perceivable after hand washing has begun, the second audible sound not perceived until the discrete event occurrence; a first fragrance not perceived until the discrete event occurrence; a first and a second fragrance, the first perceived during hand washing, the second not perceived until discrete event occurrence; or a tactility not perceivable until discrete event occurrence, including the various combinations and permutations thereof

Another specific object of the invention can be to provide hand washing agents that provide perceivable sensorial reinforcer(s) to adjust the probability of completion of a desired hand washing duration, or attainment of a level of hand washing having a desired degree of therapeutically efficacy, or the lice. As to these embodiments of the invention, perceivable sensorial reinforcer(s) can provide a color perceivable at the time hand washing begins increasing in intensity at least until generation of a perceivable sensorial indicia; a color not perceivable during hand washing until after generation of a perceivable sensorial indicia and then lasting for a duration of time substantially exceeding the period in which the perceivable sensorial indicia occurs, wherein the indicia period could have a duration of time between about 1 second to about 5 seconds (although the duration from embodiment to embodiment of the invention may vary); at least one color perceivable after hand washing begins at least until generation of the perceivable sensorial indicia; a fragrance perceivable after hand washing begins prior to generation of the perceivable sensorial indicia.

Naturally, further objects of the invention are disclosed throughout other areas of the specification and drawings.

### IV. BRIEF DESCRIPTION OF THE DRAWINGS

FIG. 1 shows an embodiment of the invention that provides a cosmetic having perceived sensorial indicia (color generation and sound generation).

FIG. 2 shows an embodiment of the invention that provides a cosmetic having a plurality of perceived sensorial indicia (heat generation and fragrance generation).

FIG. 3 shows an embodiment of the invention that provides a cosmetic having a perceived sensorial indicia (sound generation) and further includes a perceived sensorial reinforcer (color generation) to increase probability that the cosmetic use will be coincident with generation of perceivable sensorial indicia.

FIG. 4 shows an embodiment of the invention that comprises a cosmetic having a perceived sensorial indicia (generation of color 3) and further includes a plurality of perceived sensorial reinforcers (generation of color 1 and generation of color 2) to increase probability that the cosmetic will be used until discrete event occurrence.

### V. MODE(S) FOR CARRYING OUT THE INVENTION

Cosmetics that comprise, individually or in combination, elements, substances, compositions, components, or materials, that are suitable for application to the skin, hair, or nails such as soaps, shampoos, conditioners, moisturizers, masks, depilatories, lotions, creams, toothpastes, teeth whiteners, make up removers, or the like, which hold or convey aqueous activated perceivable sensorial indicia or perceivable sensorial reinforcers (whether individually or in combination) in a non-aqueous carrier.

The above-mentioned embodiments of the invention are not, however, intended to limit the use of generic aspects of the invention solely to cosmetics; and various embodiments of the invention can be useful with respect to numerous applications including, but not limited to, cleaning formulations; pharmaceutical formulations; surface preparation or finishing formulations such as automotive finish cleaners and waxes; upholstery cleaners; carpet cleaners; or the like; and should be understood to include the generic concepts which include providing a non-aqueous carrier that holds, contains, or conveys aqueous activated components; which as to certain embodiments of the invention provide at least one perceivable sensorial indicia; or which as to certain embodiments of the invention provide at least one perceivable sensorial reinforcer, or both. The description of the generic embodiments of the invention, the description of cosmetic embodiments of the invention, and the specific examples of invention set forth below provide the person of ordinary skill in the art sufficient description to make and use the numerous and varied embodiments of the invention.

Cosmetics in accordance with the invention include a substantially non-aqueous carrier. Certain embodiments of the invention can include, one or more of, ethylene glycol, polyethylene glycol, polypropylene glycol, 1, 4-butanediol, 1, 5-pentanediol, 1, 6-hexanediol, pentaeryrritol, neopentyl glycol, glycerol, sorbitol, erithrithol, methanol, ethanol, isopropanol, 1-propanol, pentanol, hexanol, phenoxyethanol, benzyl alcohol, or the like. This list is not meant to be exhaustive and a carrier in accordance with the invention can comprise any substantially non-aqueous carrier(s) which as to certain embodiments of the invention can be, individually or in combination, water miscible; water immiscible; approved for application to skin, hair, or nails (whether human or animal); or formulated for other applications, as described above. As such, this list is intended to provide a sufficient number of examples of carriers so that one of ordinary skill in the art can make and use the numerous and varied embodiments of the invention.

Cosmetics in accordance with the invention can further include a surfactant for the purpose of forming homogeneous solutions; dispersion or suspension of components or material(s); or for the purpose of creating a heterogeneous system, such as an emulsion or suspension. Surfactants can be substantially non-aqueous but water miscible for the purpose of cleaning and being free-rinsing. Surfactants may be selected from classes of anionic, cationic, nonionic, or amphoteric.

Anionic surfactants can include, but are not restricted to, sodium toluene sulfonate, sodium naphthalene sulfonate, sodium lauryl sulfate, or the like. Cationic surfactants can include, but are not limited to, alkyl, aryl or alkyl aryl amines, or the like. Nonionic surfactants can include, but are not limited to, alkyl aryl ethoxylates, alkyl ethoxylates, alkyl aryl propoxylates and alkyl propoxylates. Amphoteric surfactants having both a positive and negative charge within the same molecule can include, but are not limited to, betaine surfactants.

Hydrophile-lipophile balance (HLB) of surfactants can be important with respect to certain embodiments of the invention. Surfactants at 10 are exactly balanced between hydrophilic and lipophilic. Less than 10 becomes more lipophilic (also synonymously oleophilic). Greater than 10 becomes increasingly hydrophilic. Cosmetics encompassed by the invention typically are in the range of between about 10 to about 40. A more preferred range as to some cleaning agent embodiments of the invention may be between about 12 to about 30, and as to certain embodiments of the invention the most preferred range may be between about 15 to about 25. Again, the surfactants listed are not meant to be exhaustive, but rather are intended to provide a sufficient number of examples of the wide variety of surfactants that may be used in accordance with the invention as a means of adjusting the surface tension of liquids or water with which the invention interacts.

Certain embodiments of the invention can further include a capsule (the term “capsule” is intended to encompass any type or kind of coat, coating, wall, capsule, or encapsulating material) that can be mixed with, held, contained within, or conveyed in one or more non-aqueous carrier(s), individually or in combination, without being ruptured. The capsule can contain, or provide a surface for deposition of, solid or liquid material(s) released when exposed to aqueous materials (the term aqueous material is intended to encompass any type or kind of aqueous system, including, but not limited to, water, purified water, water containing an amount of soluble or insoluble material(s), or material(s) containing an amount of water).

Certain embodiments of the invention include capsules having a central reservoir of solid or liquid material(s) surrounded by one or more capsules made of urea-formaldehyde, various forms of gelatin, polyvinyl alcohol, polyvinyl pyrrolidone, polyvinyl methyl ether, polyvinyl emthyl ether/maleic anyhydride colpolymer, carboxy methyl cellulose, hydroxy ethyl cellulose, cellulose acetate butyrate, cellulose acetate propionate, polyvinyl alcohol/acetl copolymer, or the like. Specific non-limiting examples could include those capsules described by U.S. Pat. Nos. 2,800,457; 3,697,437; 3,336,155, each incorporated by reference herein.

The solid or liquid material(s) contained by the capsules may be aqueous miscible or non-aqueous miscible, such as surfactants, enzymes, flavors, fragrances, bleach or bleaching agents, pH change indicators, colorants, anti-statics, fabric softener, lubricants, emollients, insecticides, disinfectants, perfume, dentifrice, vaccines, drugs, medications, amino acids, nucleic acids, microbes, hormones, antiviral proteins, antiviral peptides, industrial chemicals (which includes a wide variety of materials such as oxidizing agents, reducing agents, free radical initiators, or the like), bioactive agents, lotions, gels, or the like. These numerous and varied types or kinds of solid or liquid material(s) may be further combined to impart features such as emolliency, moisturizing, lubricity, color, fragrance, texture, viscosity, or sound, so long as, the resulting solid or liquid material(s) do not cause, individually, or in combination with the carrier, the capsule to rupture.

As to certain embodiments of the invention, the capsules can be configured or constructed to allow selectably variable release of the solid or liquid material(s) after exposure to aqueous material for a duration of time. Timed release of liquid or solid material(s) can be controlled by capsule characteristics, such as, the type of capsule polymer (or other capsule material) used to generate the wall, coat, or capsule; the thickness of the coat, wall, or capsule; or the density of the coat, wall, or capsule. The range of time for release of material(s) from the capsule upon exposure to aqueous liquid can be almost instantaneous or up to about 150 seconds, although certain capsule characteristics may allow an increased duration of time to elapse prior to release of liquid or solid material(s) therein.

As such, the release of the encapsulated liquid or solid material(s) from capsules having uniform characteristics can occur within narrow window of time, or may be released over a relatively long period of time from a mixture of capsules having non-uniform capsule characteristics. The material(s) held, contained, or conveyed by certain populations of capsules having uniform characteristics can be released in a window of time of about plus two seconds or minus two seconds of a selected time after exposure to aqueous liquid. Alternatively, populations of capsules having a mixture of non-uniform capsule characteristics can provide staged release of material(s) starting at a selected time after exposure to aqueous liquid and continuing over any desired duration of time up to about 150 seconds, or beyond.

Alternative embodiments of the invention provide timed release of liquid or solid material(s) through the use of capsules within capsules. A first amount of liquid or solid material(s) can be contained in a first capsule. A second amount of liquid or solid material(s) can be absorbed, or otherwise associated with, the outside surface of the first capsule. The entirety of the first capsule containing the first amount of liquid or solid material(s) along with at least a portion of the second amount of liquid or solid material associated with the outside surface of the first capsule can be encapsulated within a second capsule. The process can be repeated a 2 or 3 times, or more, as may be desired.

Other embodiments of the invention can comprise a first capsule which does not contain any solid or liquid material(s). An amount of liquid or solid material(s) can be absorbed, or otherwise associated with, the outside surface of the first capsule. The entirety of the first empty capsule along with at least a portion of the second amount of liquid or solid material associated with the outside surface of the first capsule can be encapsulated within a second capsule.

Embodiments of the invention can provide a substantially non-aqueous carrier into which carrier stable capsules (the term “carrier stable capsules” is intended to encompass capsules that do not degrade; are not substantially degraded; sequester solid or liquid material(s) within; do not allow substantial transfer of solid or liquid materials; or do not rupture, when in contact or association with such substantially non-aqueous carrier) containing solid or liquid material(s) are mixed. The non-aqueous carriers can be used to convey carrier stable capsules for the release of solid or liquid material(s) into other non-aqueous or aqueous material(s) in which the capsule ruptures.

As to some embodiments of the invention, the composition, construction, configuration, or number, of the carrier stable capsules are selected so that release of solid or liquid material(s) therein coincide with occurrence of one or more discrete events, thus providing at least one perceivable sensorial indicia of discrete event occurrence. A non-limiting example in accordance with the invention can be a liquid hand washing agent which comprises a substantially non-aqueous carrier in which one or more populations of carrier stable capsules release solid or liquid material(s) coincide with elapse of a duration of hand washing time to notify the hand washer that hand washing can stop, or hand rising can begin.

A hand washing agent in accordance with the invention can be used by applying an amount of the hand washing agent to the surface of the hands, rubbing the hand washing agent on the surface of the hands in combination with water for about 12 seconds to about 15 seconds (although this period may vary depending upon the embodiment of the invention) until the user perceives at least one sensorial indicia that the time period has elapsed, such as generation of color or fragrance. Upon perceiving the sensorial indicia the duration of hand washing is complete and the hand washing agent can be rinsed from the surface of the hands.

An alternative to the perceivable sensorial indicia of color or fragrance, can be the perceivable sensorial indicia including auditory indicia through generation of sound waves which can be achieved through the rapid release of carbon dioxide or effervescence. A carbonate or an acid may be contained within the capsule. With respect to cleaning agents, including, but not limited to, hand washing agents, placing the carbonate in the carrier and the acidic material in a capsule may be most practical. As non-limiting examples, sodium carbonate, sodium bicarbonate; or lithium, potassium or ammonium carbonates or the bicarbonates; or other monohydrogen carbonate(s); or carbonates and bicarbonates of di- and tri-valiant alkali metal salts such as calcium, barium, iron, aluminum, or the like, can be added to the carrier composition.

Acidic material(s) such as salts of strong or weak mineral or organic acids, such as zinc chloride, sodium sulfate, monosodium phosphate, sodium acetate, or the like; or free acids such as citric acid, ascorbic acid, tartaric acid, or the like, can be contained in capsules. When the capsule releases the acidic material through rupture or dissolution, the immediate environment experiences a drop in pH causing a portion of the carbonate to convert to carbon dioxide. Expansion to a gas can cause foam generation, sound created through the breaking of the bubbles, or both. The level of sound measured in decibels can be in the range of 0.5-12 db depending upon how many CO2 bubbles simultaneously burst.

Cosmetics in accordance with the invention can further include perceivable sensorial reinforcers. Perceivable sensorial reinforcers can be utilized to increase, or in some instances decrease, the probability that the cosmetic agent will be in use when a discrete event occurs, or coincident with perceivable sensorial indicia, as described above. As such, in conjunction with, or separate from, the use of one or more perceived sensorial indicia, the invention can include the release of liquid or solid material(s) from carrier stable capsules at after a duration of time, or with a release rate which induces the user to continue use of the cosmetic to release perceivable sensorial indicia. In certain embodiments of the invention, the solid or liquid material(s) are encapsulated within a population of capsules configured or constructed selected to release perceivable sensorial reinforcers after exposure to aqueous material.

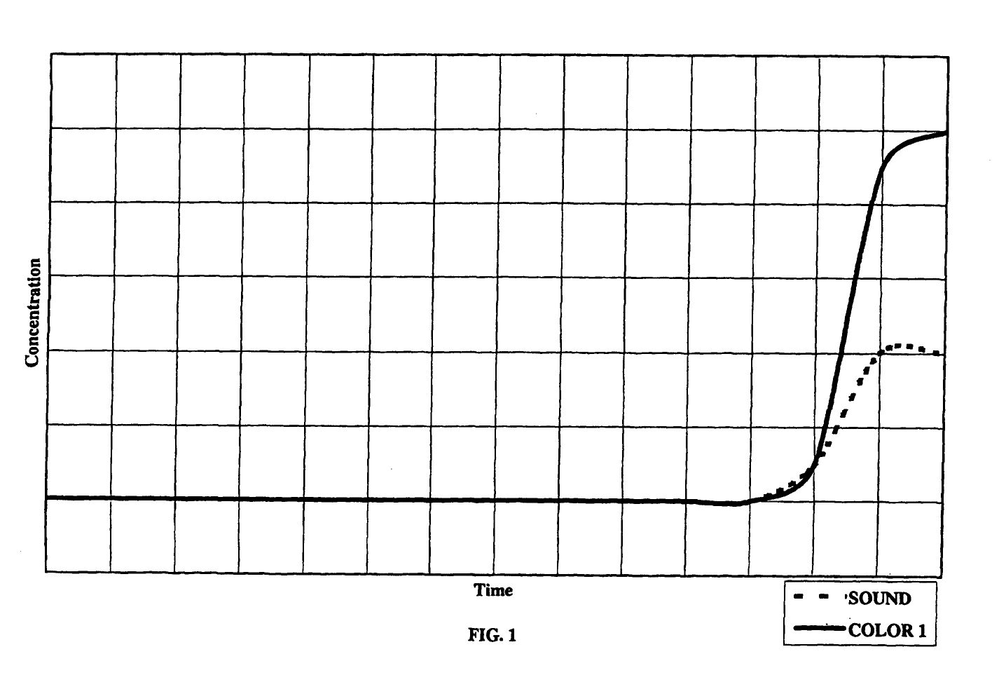

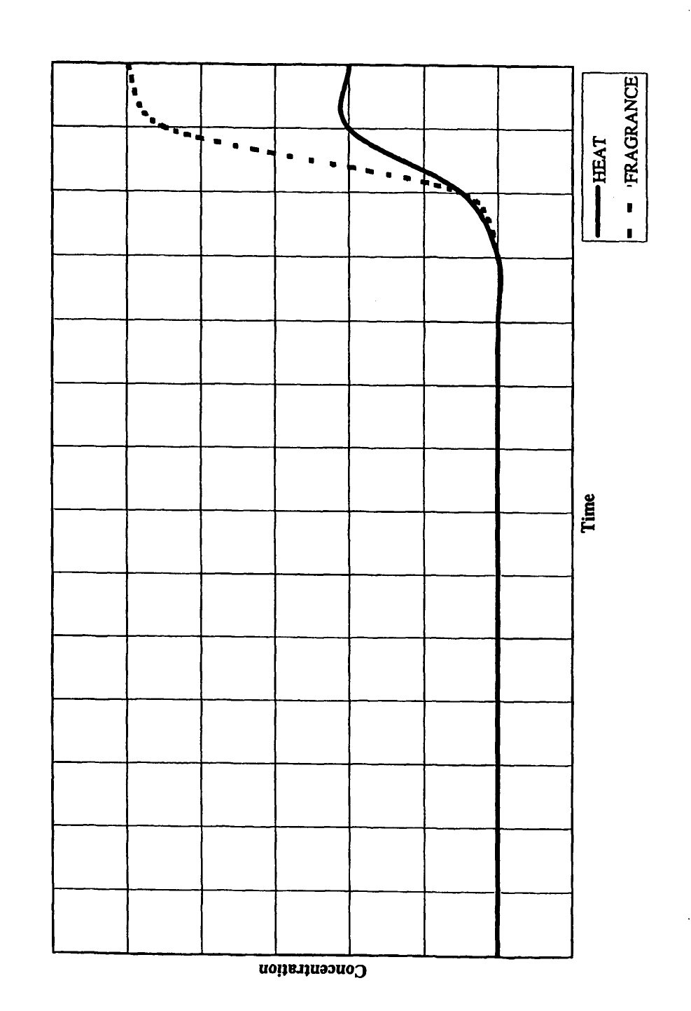

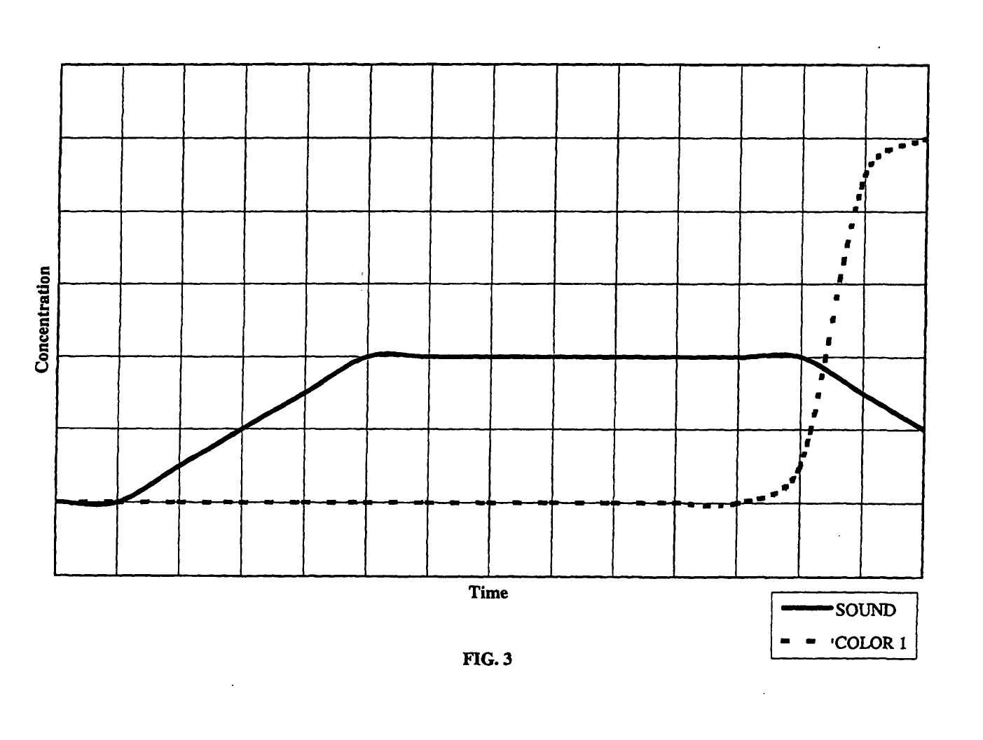

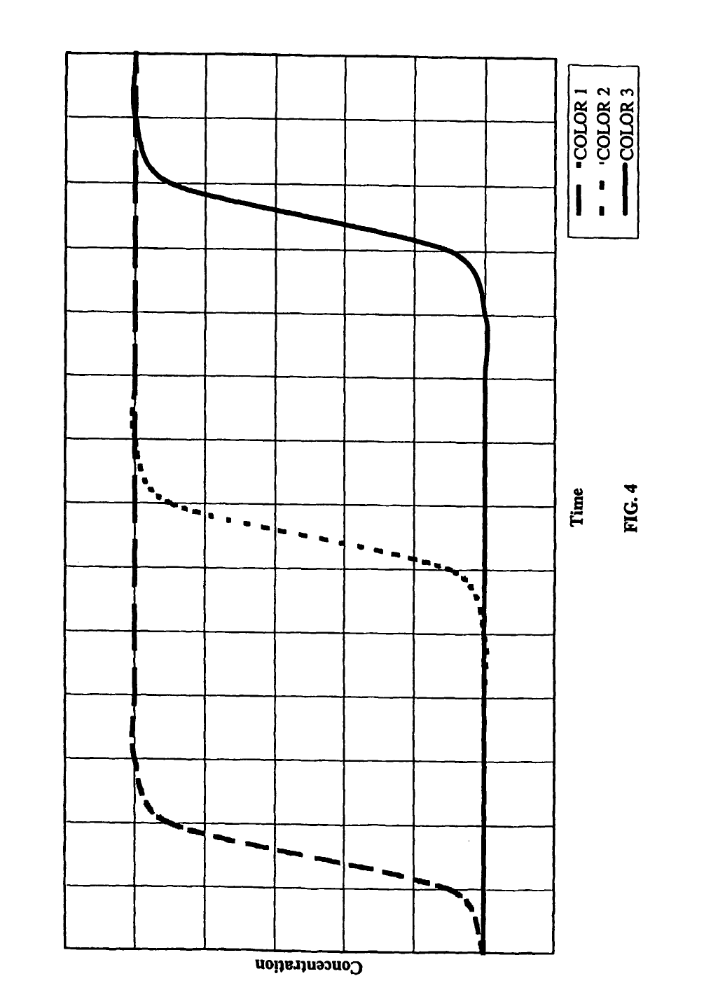

Now referring to FIGS. 1 to 4, which show non-limiting examples of cosmetics having perceived sensorial indicia and perceivable sensorial reinforcers. As can be understood from the Figures, the invention is intended to be used until a discrete event occurs which may or may not be perceivable by the cosmetic user. In the specific example of a cosmetic in accordance with the invention, the cosmetic provides at least one perceived sensorial indicia (for example, material(s) released in the aqueous material which achieve, coincident with occurrence of a discrete event, a concentration or level perceivable to the user). Moreover, individually or in combination with the provision of perceived sensorial indicia, the invention can provide perceived sensorial reinforcers (material(s) released in the aqueous material as an inducement or reward to the user to increase the probablility of achieving release of the perceivable sensorial indica).

Understandably, there are numerous combinations and permutations of perceivable sensorial indicia that can be used individually or in combination with perceivable sensorial reinforcers as described above or as shown in the Figures. As such, the examples provided by the Figures are not intended to be limiting with respect to the type or kind of cosmetic; the type or kind of use the cosmetic may be applied to; the type, kind, or duration of discrete event occurrence; the duration of time from the initial application of the cosmetic to release of the perceived sensorial indicia; the duration or concentration of the perceived sensorial indicia; the number, type or kind of perceived sensorial indicia contained or conveyed by the cosmetic; the concentration of the perceived sensorial indicia; the manner of temporal coupling of the perceived sensorial indicia to the discrete event occurrence so long as a relationship between the perceived sensorial indicia and the occurrence of the discrete event can be useful in achieving the intended consequence or proper use of the cosmetic; the number type or kind of perceived sensorial reinforcers; or the concentration or duration of the perceived sensorial reinforcer so long as the perceived sensorial reinforcer increases the probablility or decreases the probability of coincidence of cosmetic use with the occurrence of the discrete event.

The following examples of embodiments of the invention are not intended to limit the scope of the invention solely to the compositions described, but in conjunction with the description above, are intended to provide a sufficient number of examples so that a person of ordinary skill in the art would know how to make and use the numerous and wide variety of cosmetics, including the numerous embodiments of hand washing agents which provide a substantially non-aqueous carrier in which aqueous activated components are added, or those numerous embodiments of the invention that provide perceivable sensorial indicia, or those numerous embodiments of the invention that provide perceivable sensorial reinforcement attributes, individually or in combinations thereof.

### EXAMPLE 1

A composition may be prepared by adding the following components: 132 parts of glycerol, 100 parts of sodium lauryl sulfate, 20 parts of aloe vera gel, 20 parts of vitamin A gel, 20 parts of vitamin E gel and 30 parts of nonylphenoxypolyoxyethylene ethanol (12 moles EO). When blending is complete, one may thicken the mixture by adding 12 parts of an aqueous solution of polyarylamide (12% w/w) and neutralizing to a pH of 7.0+/−0.2 using a 45% solution of potassium hydroxide. A yellow dye encapsulated with polyvinyl alcohol capsule having a wall thickness allowing dissolution in about 4 seconds to about 6 seconds, and a blue dye encapsulated with polyvinyl alcohol having a wall thickness allowing dissolution in about 12 seconds to about 15 seconds may be added to the aforemention solution by adding 5 parts of the yellow capsules and 5 parts of the blue capsules. The mixture can be mixed to create a homogeneous blend.

A useful amount of the above composition may be applied to the hands with a small amount of water. The effect that may be seen is initially white foam turning yellow, and with additional time and rubbing to turn green. The hands may be rinsed with water.

### EXAMPLE 2

The composition described in Example #1 may be reproduced except that the capsule containing yellow dye is replaced with a capsule containing lime oil fragrance, also within a polyvinyl alcohol capsule.

A useful amount of this composition may be applied to the hands with the addition of a small amount of water. The effect that may be seen is initially heavy white foam followed shortly with the distinct smell of lime, and with additional time and rubbing the change from white to blue foam. The hands may be rinsed with water.

### EXAMPLE 3

In like manner as described in Example #1 a hand cleaning composition may be prepared by adding the following components: 140 parts polyethylene glycol (200), 90 parts of isooctylphenoxypolyoxyethylene ethanol (40 moles EO), 25 parts aloe vera, 20 parts of rose water in glycerin and 30 parts polyoxyethylene ethanol tridecyl ether (18 moles EO)

Once the blending is complete, one may thicken the solution by adding 8 parts of carboxy methyl cellulose. The pH is adjusted to 8.7 using the required amount of sodium carbonate. At this point one may introduce timed changes of color and sound by using an encapsulated and an encapsulated acidic compound. A red dye is encapsulated with polyvinyl pyrrolidone and having a wall thickness allowing for dissolution in 15 seconds. Sodium acetate is similarly encapsulated with polyvinyl pyrrolidone with a wall thickness allowing dissolution in 5 seconds. 5 parts of each of the two capsule types are added and mixed to create a homogeneous blend.

A useful amount of the above composition may be applied to the hands with a small amount of water. The effect that may be seen is the initial foaming and effervescent followed later by the color change to red foam. The hands may be rinsed with water.

### EXAMPLE 4

A composition may be prepared in like manner as described in Example #1 as the base solution. The color changing capability is modified to result in a three-phase color change. A yellow dye encapsulated with cyclodextrin having a wall thickness allowing dissolution in about 4 seconds to about 6 seconds, a red dye encapsulated with cyclodextrin having a wall thickness allowing dissolution in about 8 to about 10 seconds, and a blue dye encapsulated with cyclodextrin having a wall thickness allowing dissolution in about 12 to about 15 seconds may be added to the base solution. Each would be added at 5 parts to the base solution and uniformly mixed.

A useful amount of the above composition may be applied to the hands along with a small amount of the water. The effect that may be seen in initially white from turning to yellow, and then orange with the release of the red dye and finally turning purple with the release of the blue dye. The hands may be revised with water.

### EXAMPLE 5

A composition may be prepared in the like manner as described in Example #3 as the base solution. In lieu of the capsules combining a dye and an acidic compound, they may be substituted with a capsule containing a lemon oil, said capsule being comprised of polyvinyl methyl ether and having a wall thickness that allows dissolution in water in about 8 seconds to about 10 seconds, and apple fragrance encapsulated similarly in polyvinylmethyl ether and having a wall thickness that allows dissolution in water in about 12 seconds to about 15 seconds. The capsules may be added to the base solution at a level of 5 parts each and mixed until homogeneous.

A useful amount of the above composition may be applied to the hands along with a small amount of water. The effect that can be sensed is that of having essentially no smell going to a light fragrance and subsequently detecting a light apple fragrance. The odor dissipates when the hands are worked with the water. The following compositions apply to the above-described examples.

| | | |
|---|---|---|
| Carrier | Range as % (w/w) | 30-90 |
| | More Preferred | 40-85 |
| | Most Preferred | 55-80 |
| Surfactant | Range as % (w/w) | 1-60 |
| | More Preferred | 5-50 |
| | Most Preferred | 10-35 |
| Capsules | Range as % (w/w) | 0.1-10 |
| | More Preferred | 0.5-7 |
| | Most Preferred | 1.0-5 |

Other Ingredients (such as emollients, lubricity additives, moisturizing agents and the like may be optionally added).

| | |
|---|---|
| Range as % (w/w) | 0.1-20 |
| More Preferred | 1-15 |
| Most Preferred | 3-10 |

Thus, the applicant(s) should be understood to claim at least: i) each of the cosmetics as herein disclosed and described, ii) the related methods disclosed and described, iii) similar, equivalent, and even implicit variations of each of these devices and methods, iv) those alternative designs which accomplish each of the functions shown as are disclosed and described, v) those alternative designs and methods which accomplish each of the functions shown as are implicit to accomplish that which is disclosed and described, vi) each feature, component, and step shown as separate and independent inventions, vii) the applications enhanced by the various systems or components disclosed, viii) the resulting products produced by such systems or components, ix) methods and apparatuses substantially as described hereinbefore and with reference to any of the accompanying examples, x) the various combinations and permutations of each of the previous elements disclosed.

1. A cosmetic composition, comprising:
a) a non-aqueous carrier;
b) a plurality of non-aqueous carrier stable capsules;
c) an encapsulated material established within said plurality of non-aqueous carrier stable capsules;
c) at least one sensorial perceivable material generated upon release of said encapsulated material established within said plurality of non-aqueous carrier stable capsules, where in said plurality of non-aqueous carrier stable capsules rupture in an aqueous material after a duration of time.

2. A cosmetic composition as described in claim 1, wherein said plurality of non-aqueous carrier stable capsules comprise a coat of polyvinyl alcohol.

3. A cosmetic composition as described in claim 1, wherein said plurality of non-aqueous carrier stable capsules comprise a coat of polyvinyl pyrrolidone.

4. A cosmetic composition as described in claim 1, wherein said plurality of non-aqueous carrier stable capsules comprise a coat of cyclodextrin.

5. A cosmetic composition as described in claim 1, wherein said plurality of non-aqueous carrier stable capsules comprise a coat of polyvinylmethyl ether.

6. A cosmetic composition as described in claim 1, wherein said at least one encapsulated material comprises a dye.

7. A cosmetic composition as described in claim 6, wherein said at least one dye is selected from the group consisting of azo dye, disazo dye, diazonium salts, azoxy dye, hydrazo dye, benzidine dye, anthraquinone dye, triphenylmethane dye, aniline dye, aquonium dye, cationic dye, chrome dye, fluorescent dye, leuco dye, nitro dye, naphthol dye, toluidine dye, acridine dye, tetrakisazo dye, thiazole dye, fluorine dye, oxazin dye, tetraazolium salt dye, thiazin dye, pyronin dye, rhodamine dye, safranin dye, phthalocyanin dye, acid dye, base dye, and substituted analogs thereof.

8. A cosmetic composition as described in claim 1, wherein said at least one encapsulated material comprises an indicator which changes color based upon pH.

9. A cosmetic composition as described in claim 8, wherein said indicator which changes color based upon pH is selected from the group consisting of alzarin red, bromcresol green, bromcresol purple, bromcresol blue, congo red, crystal violet, dimethyl yellow, ethyl violet, malachite green, m-cresol purple, ntanil yellow, methyl orange, methyl purple, methyl red, methyl violet, phenol red, resorcin blue, and thymol blue, brilliant yellow, bromthylmol blue, m-nitrophenol, neutral red, phenophtalein, alizarin red S, alkali blue, clayton yellow, cresol red, curcumin, m-cresol purple, o-cresophthalein, phenol violet, p-naphtholbenzein, thymol violet, thymolphtalein, and titan yellow.

10. A cosmetic composition as described in claim 1, wherein said encapsulated material comprises a fragrance.

11. A cosmetic composition as described in claim 1, wherein said encapsulated material comprises a base.

12. A cosmetic composition as described in claim 11, wherein said base is selected from the group consisting of sodium carbonate, sodium bicarbonate, lithium carbonate, potassium carbonate, ammonium carbonates, sodium bicarbonate, lithium bicarbonate, and trisodium phosphate.

13. A cosmetic composition as described in claim 1, wherein said encapsulated material comprises an acid.

14. A cosmetic composition as described in claim 13, wherein said acid is selected from the group consisting of zinc chloride, sodium sulfate, monosodium phosphate, sodium acetate, citric acid, ascorbic acid, and tartaric acid.

15. A cosmetic composition as described in claim 14, wherein said non-aqueous carrier contains an amount of base.

16. A cosmetic composition as described in claim 15, wherein said base is selected from the group consisting of wherein said base is selected from the group consisting of sodium carbonate, sodium bicarbonate, lithium carbonate, potassium carbonate, ammonium carbonates, sodium bicarbonate, lithium bicarbonate, and trisodium phosphate.

17. A cosmetic composition as described in claim 12, wherein said non-aqueous carrier further comprises an indicator which changes color based upon pH.

18. A cosmetic composition as described in claim 17, wherein said indicator which changes color based upon pH is selected from the group consisting of alzarin red, bromcresol green, bromcresol purple, bromcresol blue, congo red, crystal violet, dimethyl yellow, ethyl violet, malachite green, m-cresol purple, ntanil yellow, methyl orange, methyl purple, methyl red, methyl violet, phenol red, resorcin blue, and thymol blue, brilliant yellow, bromthylmol blue, m-nitrophenol, neutral red, phenophtalein, alizarin red S, alkali blue, clayton yellow, cresol red, curcumin, m-cresol purple, o-cresophthalein, phenol violet, p-naphtholbenzein, thymol violet, thymolphtalein, and titan yellow.

19. A cosmetic composition as described in claim 1, wherein said non-aqueous carrier comprises a mixture of glycerol, sodium lauryl sulfate, and aleo vera gel.

20. A cosmetic composition as described in claim 18, wherein said non-aqueous carrier comprises a mixture of polyethylene glycol.

21. A cosmetic composition as described in claim 1, wherein said plurality of non-aqueous carrier stable capsules rupture after a duration of time in aqueous material to release said encapsulated material contained within such non-aqueous carrier stable capsules to generate a perceived sensorial indicia coincident to a discrete event occurrence.

22. A cosmetic composition as described in claim 21, wherein said discrete event occurrence comprises attainment of a level of therapeutic efficacy.

23. A cleaning composition as described in claim 21, wherein said discrete event occurrence comprises compliance with regulatory guidelines for use of said cleaning composition.

24. A cleaning composition as described in claim 21, wherein said cleaning composition comprises a liquid hand soap and wherein said discrete event occurrence comprises expiration of a duration of time in compliance with hand washing procedure.

25. A cleaning composition as described in claim 24, wherein said duration of time comprises a duration of time between about 5 seconds and about 15 seconds.

26. A cleaning composition as described in claim 21, further comprising perceivable sensorial reinforcement attributes.

27. A cleaning composition as described in claim 1, wherein said capsules are selected from the group consisting of: single capsule type, capsule within capsule type, a mixture of different configurations of single capsule types, and a mixture of different configurations of capsule within capsule types.

28. A method of washing hands, comprising the steps of:
a) applying a liquid hand soap to hands, wherein said liquid soap comprises a non-aqueous carrier containing a plurality of capsules that rupture after a duration of time when exposed to water;
b) combining said liquid hand soap applied to said hands with water;
c) rubbing said liquid hand soap applied to said hands; and
d) perceiving sensorial attributes of substances released from said capsules that rupture.

29. A method of washing hands as described in claim 11, further comprising the step of configuring said capsules to rupture after a duration of time coincident to a discrete event occurrence.

30. A method of washing hands as described in claim 11, further comprising the step of configuring a population of capsules to rupture at a rate which provides perceivable sensorial reinforcement attributes.
